(19) United States
(12) Patent Application Publication
(10) Pub. No.: US 2026/0142033 A1
(43) Pub. Date: May 21, 2026

(54) NEURAL NETWORK AUTOMATED INVASIVE ARTERIAL PRESSURE EXTRACTION

(71) Applicant: REGENTS OF THE UNIVERSITY OF MICHIGAN, Ann Arbor, MI (US)

(72) Inventors: Zachary Sharpe, Ann Arbor, MI (US); Mohamad Hakam Tiba, Ann Arbor, MI (US); Cindy H. Hsu, Ann Arbor, MI (US)

(21) Appl. No.: 19/387,810

(22) Filed: Nov. 13, 2025

Related U.S. Application Data
(60) Provisional application No. 63/721,211, filed on Nov. 15, 2024.

(51) Int. Cl. G16H 50/20; A61H 31/00; A61K 31/137; A61K 38/095

(52) U.S. Cl. CPC G16H 50/20; A61H 31/005; A61K 31/137; A61K 38/095; A61H 2201/105; A61H 2201/501; A61H 2201/5097; A61H 2230/305

(57) ABSTRACT

The methods and systems described herein relate to automatically analyzing arterial pressure waveforms during cardiopulmonary resuscitation using neural networks and other machine learning models or processing methods. The methods and systems may differentially label diastolic and systolic blood pressure coming from chest compression-induced arterial waveforms versus spontaneous heartbeats. In one aspect, a portable device receives arterial pressure data in real time and makes predictions of the systolic pressure and/or diastolic pressure using an on-board machine learning model.

### CROSS-REFERENCE TO RELATED APPLICATIONS

This application claims the benefit of U.S. Application No. 63/721,211, filed November 15, 2024, and entitled “Neural Network Automated Invasive Arterial Pressure Extraction”, which is incorporated herein by reference in its entirety.

### STATEMENT OF GOVERNMENT SUPPORT

This invention was made with government support under 5 R61 NS123760-02 awarded by the National Institutes of Health. The government has certain rights in the invention.

### FIELD OF THE INVENTION

The present disclosure generally relates to accurately measuring arterial blood pressure properties during cardiopulmonary resuscitation (CPR) and spontaneous heartbeat, and more particularly to automatically analyzing arterial pressure waveforms using neural networks and other machine learning models or processing methods.

### BACKGROUND

Cardiac arrest is the inability of the heart to adequately pump blood in a life-sustaining manner due to several possible causes. CPR is the manual pumping of blood by compressing the chest during cardiac arrest, often performed with automated mechanical devices to minimize rescuer fatigue. The diastolic blood pressure, or the resting pressure in the arteries between heartbeats or compressions, is a helpful measurement during CPR from which to extrapolate coronary perfusion pressure (CPP). CPP during cardiac arrest is the difference between aortic diastolic pressure and right atrial diastolic pressure but may be best conceptualized as diastolic blood pressure–central venous pressure. CPP is the principal contributor to myocardial blood flow during CPR; thus, having a sufficient CPP is a key determinant in resuscitation outcome.

Due to changes in how CPR drive blood flow, the shape of waveforms measured on invasive arterial pressure lines changes, which can lead to inaccurate identification of diastolic blood pressure. The active compression-decompression method of CPR can cause negative deflections on the arterial pressure waveform. This negative deflection may cause incorrect diastolic blood pressure detection when trying to use standard peak/local minima detection to analyze arterial pressure data post-cardiac arrest.

There exists a need for fast and accurate techniques to automatically detect diastolic blood pressure during CPR, and in particular, to distinguish a spontaneous (i.e., heartbeat) diastolic event from a compression diastolic event in a waveform.

### SUMMARY

The following relates to systems and methods for automatically analyzing arterial pressure waveforms during chest compressions using neural networks and other machine learning models or processing methods. The techniques disclosed herein may differentially label diastolic and systolic blood pressure coming from chest compression-induced waveforms versus spontaneous heartbeats.

In one aspect, the techniques disclosed herein are implemented by a machine learning model, such as, for example a compressed, fully-convolutional network (FCN). In other aspects, the techniques are implemented by U-Net, a gated recurrent unit (GRU), a long short-term memory (LSTM) model or any appropriate machine learning model or combinations of machine learning/neural network models or other processing methods. In one aspect, the FCN, GRU, and/or LSTM comprise stacked dilated convolutional layers.

In one aspect, the techniques include a portable computing device that connects directly to a patient monitor. The portable computing device receives arterial pressure data in real time and makes predictions of the diastolic pressure and/or CPP using an on-board neural network model. The portable computing device may include a display, which may output a systolic/diastolic number and/or a pressure waveform. In another aspect, the neural network model implementing the techniques may be integrated into the patient monitor device itself.

In one aspect, the techniques disclosed herein include receiving sampled arterial blood pressure data points and labeling the data points with the following categories: nothing, compression diastolic, compression systolic, spontaneous diastolic, and/or spontaneous systolic.

In one aspect, a computer-implemented method of determining arterial blood pressure may be provided. The method may include: (1) providing a blood pressure input vector comprising a plurality of blood pressure measurements of a patient during a time period to a machine learning model, causing the machine learning model to: (a) normalize the plurality of blood pressure measurements into a normalized blood pressure vector, (b) convolve, using a first set of one or more filters, the normalized blood pressure vector into a first feature map, (c) convolve, using a second set of one or more dilated filters, the first feature map into a second feature map, wherein the second set of one or more dilated filters are dilated at a first dilation rate, (d) convolve, using a third set of one or more dilated filters, the second feature map into a third feature map, wherein the third set of one or more dilated filters are dilated at a second dilation rate that is greater than the first dilation rate, (e) convolve, using a fourth set of one or more dilated filters, the third feature map into a probability matrix, wherein the probability matrix comprises probabilities for a plurality of classifications for one or more of the plurality of blood pressure measurements, wherein the plurality of classifications comprise spontaneous systolic pressure and spontaneous diastolic pressure, and (f) generate, from the probability matrix, an output vector comprising indications of predicted classifications for the one or more of the plurality of blood pressure measurements; and (2) outputting, based on the output vector, an indication of the spontaneous systolic pressure and the spontaneous diastolic pressure to a user. The method may include additional, less, or alternate functionality or actions, including those discussed elsewhere herein.

In one aspect, a portable computing device for determining arterial blood pressure may be provided. The portable computing device may include: (A) one or more processors; and (B) one or more non-transitory memories coupled to the one or more processors and storing instructions that when executed by the one or more processors, cause the one or more processors to: (1) provide a blood pressure input vector comprising a plurality of blood pressure measurements of a patient during a time period to a machine learning model, causing the machine learning model to: (a) normalize the plurality of blood pressure measurements into a normalized blood pressure vector, (b) convolve, using a first set of one or more filters, the normalized blood pressure vector into a first feature map, (c) convolve, using a second set of one or more dilated filters, the first feature map into a second feature map, wherein the second set of one or more dilated filters are dilated at a first dilation rate, (d) convolve, using a third set of one or more dilated filters, the second feature map into a third feature map, wherein the third set of one or more dilated filters are dilated at a second dilation rate that is greater than the first dilation rate, (e) convolve, using a fourth set of one or more dilated filters, the third feature map into a probability matrix, wherein the probability matrix comprises probabilities for a plurality of classifications for one or more of the plurality of blood pressure measurements, wherein the plurality of classifications comprise spontaneous systolic pressure and spontaneous diastolic pressure, and (f) generate, from the probability matrix, an output vector comprising indications of predicted classifications for the one or more of the plurality of blood pressure measurements, and (2) output, based on the output vector, an indication of the spontaneous systolic pressure and the spontaneous diastolic pressure to a user. The portable computing device may include additional, less, or alternate functionality, including that discussed elsewhere herein.

In one aspect, a computer-implemented method of training a machine learning model to predict systolic blood pressure and diastolic blood pressure may be provided. The method may include (1) providing a labeled training dataset to one or more machine learning models, wherein the labeled training dataset comprises a plurality of blood pressure data points and a plurality of labeled classifications; (2) receiving predicted classification outputs from the one or more machine learning models; (3) calculating a loss metric by comparing the predicted classification outputs to the plurality of labeled classifications; (4) adjusting, using the loss metric, one or more weights and/or biases of the of one or more machine learning models; and (5) compressing, by the one or more processors, a selected one of the one or more machine learning models into a compressed machine learning model. The method may include additional, less, or alternate functionality or actions, including those discussed elsewhere herein.

One of the primary improvements introduced by the methods and systems disclosed herein is an improvement to computer technology via a reduction in processor usage, memory usage, and storage footprint. Conventional neural network models have high processor, memory, and storage requirements. These requirements impose a minimum hardware requirement for performing near real-time neural network waveform analysis. The present techniques address this hardware requirement issue by compressing the model, e.g., using quantization to reduce parameters from 64 floating point bits to lower floating point bits, such as 32 bits, 16 bits or 8 bits, and using stacked dilated convolutional layers to increase the responsive window size without increasing the filter size. The present techniques reduce inference time to, for example, 50 milliseconds, and reduce model size by a factor of eight or more without significantly affecting accuracy. These quantization and stacked dilated convolution approaches decrease the processor usage, memory usage, and storage footprint of the neural network model, thus allowing a portable computing device, such as a microcomputer or microcontroller, to execute the machine learning model. For example, a compressed stacked dilated convolutional model running on a Raspberry Pi 4 microcomputer was able to run inference on a two second input data segment and return output results in roughly 50 milliseconds. These portable computing devices may then be deployed in a non-clinical or non-research setting, such as an ambulance.

Furthermore, the methods and systems reduce network usage in distributed computing environments. The techniques disclosed herein enable fast and accurate waveform analysis and arterial blood pressure determination at an endpoint device. This determination at the endpoint devices reduces or eliminates the need for a centralized server to perform the determination, which in turn reduces or eliminates the need to communicate waveform input data and blood pressure prediction output data in real time over a network.

The methods and systems disclosed herein represent an improvement to an existing technology or technologies, specifically measuring diastolic blood pressure. No technologies currently exist that can accurately and automatically measure blood pressure measurement during CPR.

In summary, the methods and systems introduce an innovative approach to determining arterial blood pressure during CPR and spontaneous heartbeat. By leveraging machine learning models or other processing methods, these techniques offer a fast and accurate solution to a challenging problem in medicine. The improvements in processor, memory, and storage usage represent a substantial advancement over existing methods and systems, opening new avenues for research and application in various fields.

Additional, alternate and/or fewer actions, steps, features and/or functionality may be included in one aspect and/or embodiments, including those described elsewhere herein.

### BRIEF DESCRIPTION OF THE DRAWINGS

The figures described below depict various aspects of the methods and systems disclosed herein. It should be understood that each figure depicts one embodiment of a particular aspect of the disclosed systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 depicts a block diagram of an exemplary blood pressure analysis system in which techniques for analyzing arterial pressure waveforms are implemented, according to some aspects.

FIG. 2 depicts exemplary arterial pressure waveforms, according to some aspects.

FIG. 3 depicts a flow diagram of an exemplary method for processing training data sets and validation data sets, according to some aspects.

FIG. 4 depicts a block diagram of exemplary machine learning model training, according to some aspects.

FIG. 5A depicts a block diagram of an exemplary stacked dilated fully convolutional network for analyzing arterial pressure waveforms, according to some aspects.

FIG. 5B depicts a block diagram of exemplary normal and dilated convolutions, according to some aspects.

FIG. 6 depicts an exemplary display for a blood pressure analysis device, according to some aspects.

FIG. 7 depicts a flow diagram of an exemplary method for predicting blood pressure, according to some aspects.

FIG. 8 depicts a flow diagram of an exemplary method for training a model to predict blood pressure, according to some aspects.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

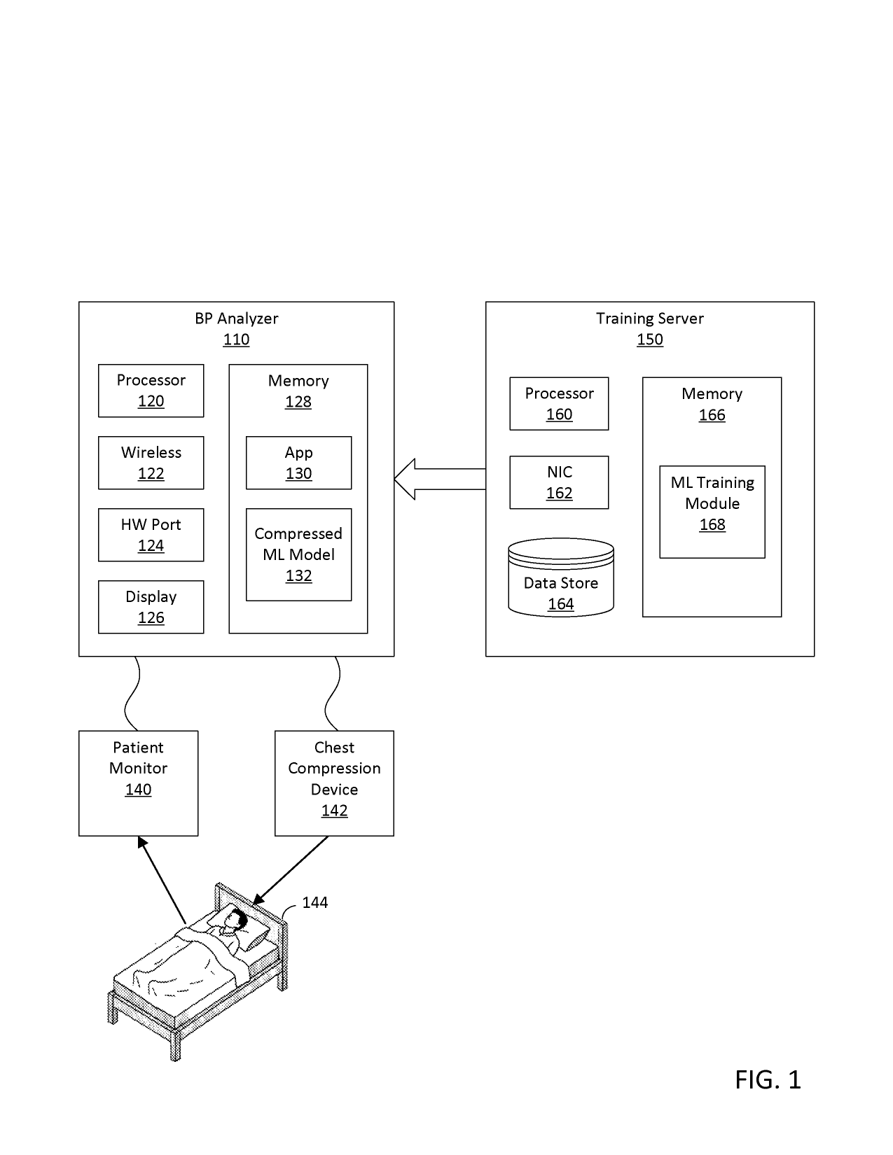

FIG. 1 depicts a block diagram of an exemplary blood pressure analysis system 100 in which techniques for analyzing arterial pressure waveforms during chest compressions using neural networks or other machine learning or processing methods may be performed, in accordance with various aspects discussed herein. As illustrated, the exemplary blood pressure analysis system 100 includes a blood pressure analyzer 110, patient monitor 140, chest compression device 142, patient 144, and training server 150. Although FIG. 1 depicts certain entities, components, equipment, and devices, it should be appreciated that additional or alternate entities, components, equipment, and devices are also possible. Of course, it should be appreciated that, while the various components of the exemplary blood pressure analysis system 100 (e.g., a blood pressure analyzer 110, patient monitor 140, chest compression device 142, patient 144, and training server 150, etc.) are illustrated in FIG. 1 as single components, the exemplary blood pressure analysis system 100 may include a plurality of blood pressure analyzers 110, patient monitors 140, chest compression devices 142, patients 144, and/or training servers 150. In some embodiments, one or more components of the exemplary blood pressure analysis system 100 (e.g., training server 150, etc.) are located in a remote data center, such as a cloud computing environment. It should be noted that the patient may alternatively receive chest compression manually.

As described herein and in an aspect, the blood pressure analyzer 110 includes a computing device, such as a server, laptop, desktop, smartphone, tablet, or any other suitable computing device. In some embodiments, the blood pressure analyzer 110 includes a Raspberry Pi, Arduino, or ESP32 microcomputer or microcontroller. In another embodiment, the blood pressure analyzer is integrated into the patient monitor 140 or chest compression device 142.

As described herein and in an aspect, the blood pressure analyzer 110 includes a processor 120. The processor 120 may include one or more suitable processors (e.g., central processing units (CPUs) and/or graphics processing units (GPUs)). The processor 120 may be connected to the memory 128 via a computer bus (not depicted) responsible for transmitting electronic data, data packets, or otherwise electronic signals to and from the processor 120 and memory 128 in order to implement or perform the machine-readable instructions, methods, processes, elements or limitations, as illustrated, depicted, or described for the various flowcharts, illustrations, diagrams, figures, and/or other disclosure herein. The processor 120 may execute an operating system (OS) and/or computing instructions contained therein.

As described herein and in an aspect, the blood pressure analyzer 110 includes a wireless interface 122. The wireless interface 122 may enable communication via cellular, ZigBee, Bluetooth, IEEE 802.11, or other suitable wireless network technologies. The blood pressure analyzer 110 may communicate with the patient monitor 140, chest compression device 142, and/or training server 150 via the wireless interface 122.

As described herein and in an aspect, the blood pressure analyzer 110 includes a hardware port 124. The hardware port 124 may be an Ethernet, USB, USB-C, Lightning, or any other suitable port. The hardware port 124 may enable the blood pressure analyzer 110 to be communicatively connected to the patient monitor 140 and/or the chest compression device 142 via one or more cables.

As described herein and in an aspect, the blood pressure analyzer 110 includes a display 126. The display may include a light-emitting diode (LED), liquid crystal display (LCD), and/or any other suitable display technology. The display 126 may depict a blood pressure waveform, systolic and diastolic blood pressure, and/or any other suitable information. The display 126 may include a graphical user interface (GUI) for controlling and/or monitoring the blood pressure analyzer 110.

As described herein and in an aspect, the blood pressure analyzer 110 includes a memory 128. The memory 128 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 128 may store an operating system (OS) (e.g., Linux, iOS, Android, etc.) capable of facilitating the functionalities, apps, methods, or other software as discussed herein.

The memory 128 may store a plurality of computing modules, such as the application 130 and the compressed machine learning (ML) model 132, implemented as respective sets of computer-executable instructions (e.g., one or more source code libraries, ML models, input/output modules, etc.) as described herein. In general, a computer program or computer-based product, application, or code (e.g., the model(s), such as ML models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor 120 (e.g., working in connection with the respective operating system in memory 128) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As described herein and in an aspect, the memory 128 includes an application 130. The application 130 may receive data from the patient monitor 140 and/or output instructions to the chest compression device 142. The application 130 may receive user input and/or provide user output via the display 126. The application 130 may provide one or more inputs to and receive one or more outputs from the compressed ML model 132.

As described herein and in an aspect, the memory 128 includes a compressed ML model 132. The compressed ML model 132 may include a trained neural network ML model, such as FCN, U-Net, GRU, or LSTM. The compressed ML model 132 may receive as input a plurality of blood pressure measurements. The compressed ML model 132 may categorize one or more of the plurality of blood pressure measurements into a plurality of categories, such as nothing, compression diastolic, compression systolic, spontaneous diastolic, and/or spontaneous systolic.

As described herein and in an aspect, the patient monitor 140 includes a patient bedside monitor, such as the Edwards Acumen IQ sensor, that generates invasive arterial pressure data. The patient monitor 140 may include an intra-arterial cannula, a fluid-filled tubing, a pressure transducer, and a processor.

As described herein and in an aspect, the chest compression device 142 includes a Lund University Cardiopulmonary Assist System (LUCAS) or another mechanical CPR device that provides mechanical chest compressions to patients in cardiac arrest.

As described herein and in an aspect, the training server 150 may generate, train, validate, and/or update the compressed ML model 132. The training server 150 may include one or more processors 160, network interface cards (NICs) 162, data stores 164, and/or memories 166.

As described herein and in an aspect, the processor 160 may include one or more suitable processors (e.g., CPUs and/or GPUs). The processor 160 may be connected to the memory 166 via a computer bus (not depicted) responsible for transmitting electronic data, data packets, or otherwise electronic signals to and from the processor 160 and memory 166 in order to implement or perform the machine-readable instructions, methods, processes, elements or limitations, as illustrated, depicted, or described for the various flowcharts, illustrations, diagrams, figures, and/or other disclosure herein. The processor 160 may execute an operating system (OS) and/or computing instructions contained therein.

The NIC 162 may include any suitable network interface controller(s), such as wired/wireless controllers (e.g., Ethernet controllers), and facilitate bidirectional/ multiplexed networking over one or more local area networks and/or wide area networks between the training server 150 and other components of the exemplary blood pressure analysis system 100 (e.g., the blood pressure analyzer 110, etc.).

The training server 150 may include or have access to the data store 164. The data store 164 may include a relational database, such as Oracle, DB2, MySQL, a NoSQL based database, such as MongoDB, or another suitable database. The data store 164 may store training data that is used to train and/or validate one or more ML models.

The memory 166 may include one or more forms of volatile and/or non-volatile, fixed and/or removable memory, such as ROM, EPROM, RAM, EEPROM, and/or other hard drives, flash memory, MicroSD cards, and others. The memory 166 may store an operating system (OS) (e.g., Microsoft Windows, Linux, UNIX, MacOS, etc.) capable of facilitating the functionalities, apps, methods, or other software as discussed herein.

In one aspect, the store an ML training module (MLTM) 168. The MLTM 168 may be included as a library or package executed on the training server 150. For example, libraries may include the TensorFlow based library, the Numpy library, the PyTorch library, the HuggingFace library, and/or the scikit-learn Python library.

In one embodiment, the MLTM 168 employs supervised learning, which involves identifying patterns in existing data to make predictions about subsequently received data. Specifically, the ML model is “trained” (e.g., via MLTM 168) using training data, which includes example inputs and associated example outputs. Based upon the training data, the ML model may generate a predictive function which maps outputs to inputs and may utilize the predictive function to generate ML outputs based upon data inputs. The exemplary inputs and exemplary outputs of the training data may include any of the data inputs or ML outputs described above. In the exemplary embodiments, a processing element may be trained by providing it with a large sample of data with known characteristics or features.

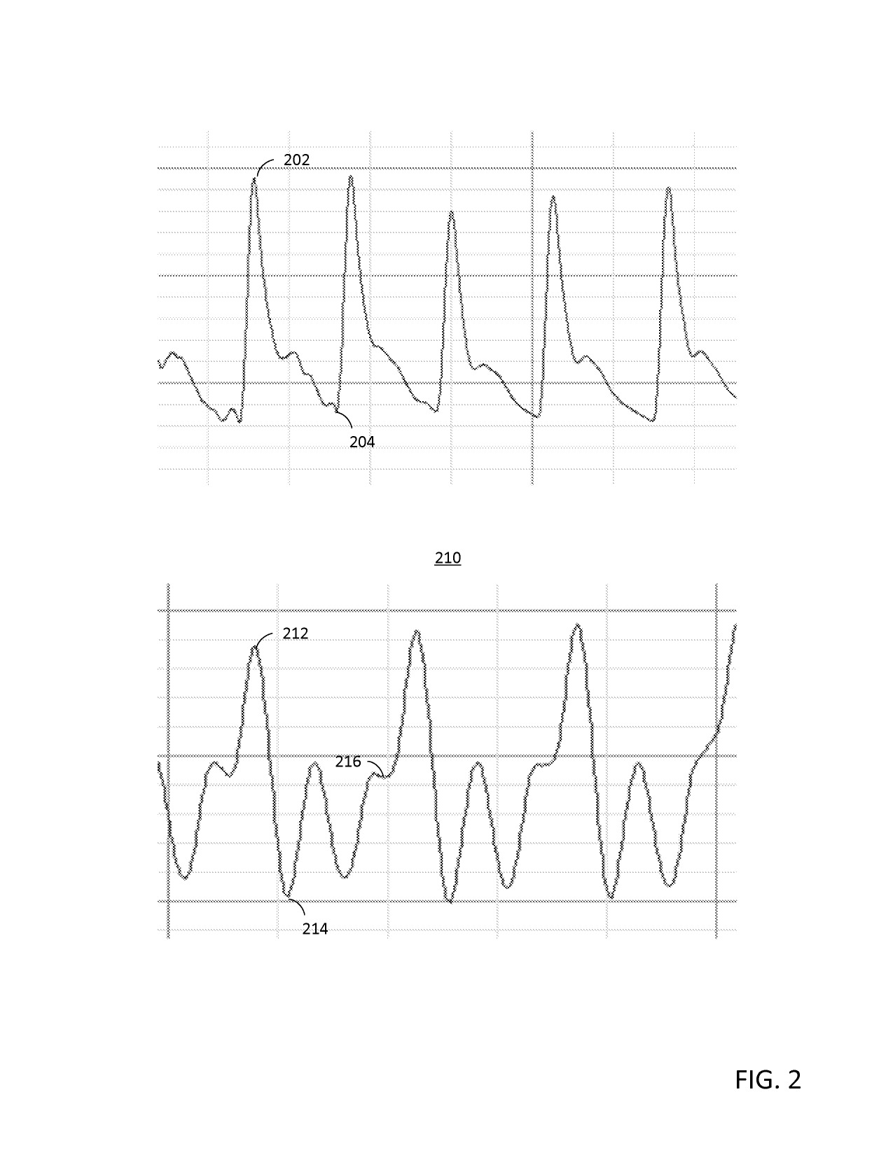

FIG. 2 depicts exemplary arterial pressure waveforms that illustrate blood pressure over time during the course of a plurality of heartbeat or chest compression cycles.

The exemplary spontaneous waveform 200 illustrates an example of arterial pressure during a spontaneous (i.e., normal) heartbeat pattern. The systolic pressure 202 corresponds to the local maximum and the diastolic pressure 204 corresponds to the local minimum of the exemplary spontaneous waveform 200.

The exemplary compression waveform 210 illustrates an example of arterial pressure during chest compression, such as manually or machine-delivered CPR. The systolic pressure 212 corresponds to the local maximum, and the diastolic pressure 216 of the exemplary compression waveform 210 corresponds to the local minimum of the exemplary spontaneous waveform 200. But algorithms, such as those employed by existing hemodynamic monitors, may incorrectly identify local minimum 214 of the exemplary compression waveform 210 as the true compression diastolic pressure.

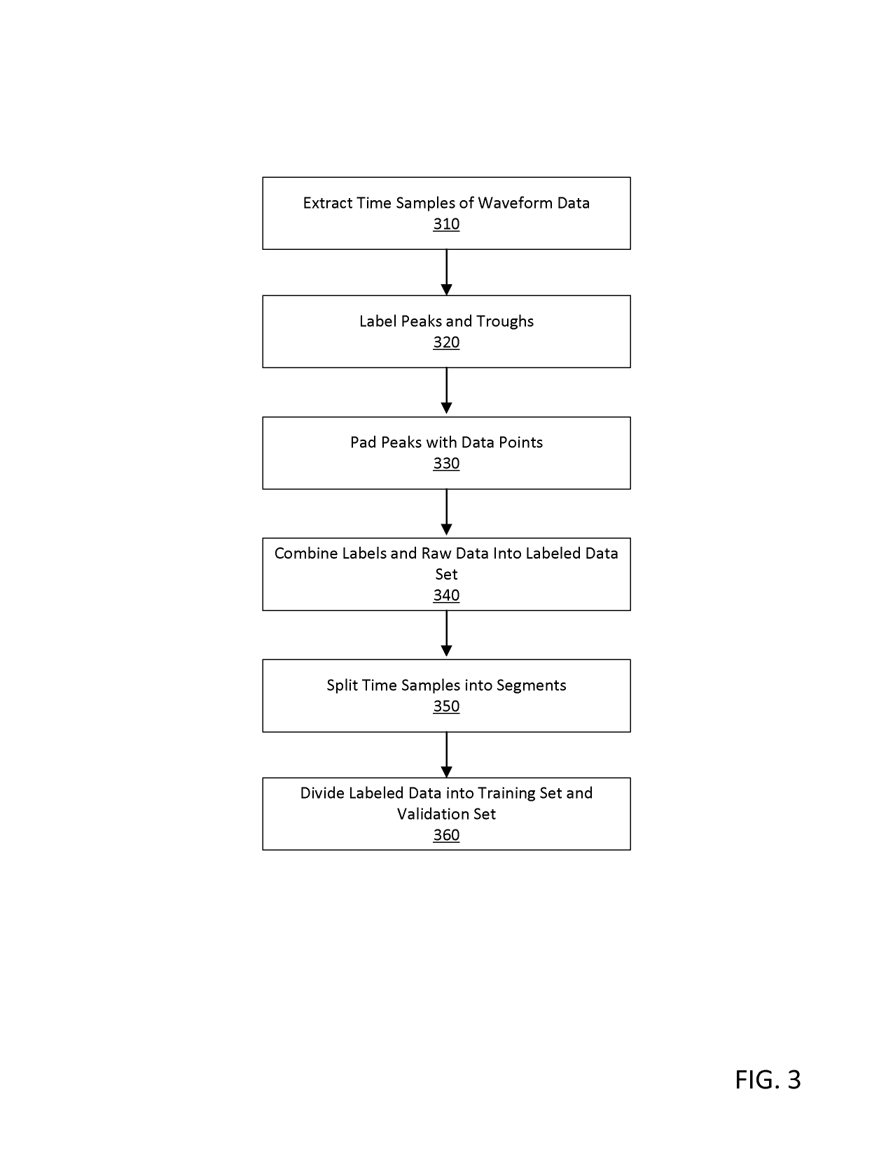

FIG. 3 depicts a flow diagram of an exemplary method 300 for generating a training data set and a validation data set from waveform data, in accordance with various aspects discussed herein. One or more steps of the method 300 may be implemented as a set of instructions stored on a computer-readable memory and executable on one or more processors. The method 300 of FIG. 3 may be implemented via a system, such as the training server 150.

In some aspects, the method 300 may include at block 310 extracting a plurality of time samples of arterial pressure waveform data. The time samples may be a fixed duration, e.g., 20 seconds. The arterial pressure waveform data may be sampled at a rate of 200 Hz, for example. The arterial pressure waveform data may include data from one or more experiments or observations. The arterial pressure waveform data may include experimental animal data, e.g., swine, and/or observational human data. The arterial pressure waveform data may include spontaneous heartbeat and cardiac arrest data.

In some aspects, the method 300 may include at block 320 labeling peaks in the time samples. The labeling may include labeling the maximum value in a labeled region of a waveform as the systolic pressure. The labeling may include labeling the value at the center of a labeled region as the diastolic pressure. The labeling may include distinguishing between spontaneous pressure and compression pressure. The labeling may be performed with the assistance of a software tool, such as MATLAB, and/or manual labeling.

In some aspects, the method 300 may include at block 330 padding labeled peaks in the time samples with additional positive labels to increase network sensitivity. In some embodiments, four to five data points are added before and after the labeled peak, which may be a single data point, such that five to six consecutive data points may be labeled as spontaneous diastolic, for example.

In some aspects, the method 300 may include at block 340 combining the labels and the raw waveform data together into a labeled data set.

In some aspects, the method 300 may include at block 350 splitting the time samples of the labeled data set into a plurality of segments. The segments may be a fixed length, e.g., two seconds.

In some aspects, the method 300 may include at block 360 dividing the labeled data set into a training data set and a validation data set. In some embodiments, the labeled data set may be randomly divided such that the training data set includes 80% of the labeled data set and the remaining 20% of the labeled data set is reserved for the validation data set. The labeled data set may be divided into the training data set and the validation data set using k-fold, e.g., 5-fold, cross-validation. In k-fold cross-validation, the labeled data set is shuffled randomly, then split into k folds (groups). Each fold is used as the validation set in one turn while the remaining k-1 folds are used for training, and the train/validate process is repeated k times.

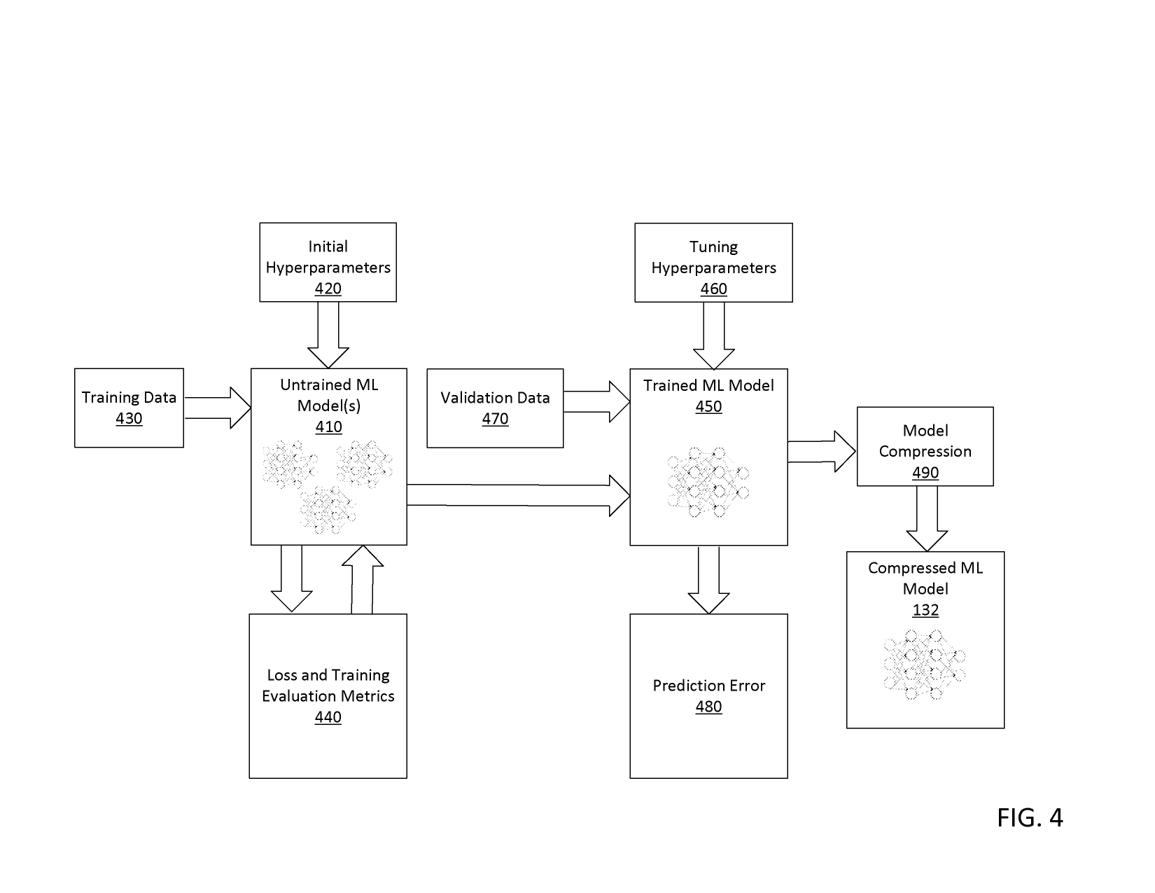

FIG. 4 illustrates an exemplary machine learning (ML) environment 400 for ML training and validation, in accordance with various aspects discussed herein. The ML training and validation may be performed by the MLTM 168 or by any other suitable code or software.

In some embodiments, there may be one or more untrained ML models 410. The untrained ML models 410 may include one or more neural network ML techniques, including FCN, U-Net, GRU, or LSTM.

The untrained ML models 410 may be configured with a set of initial hyperparameters 420. For an FCN model, for example, the set of initial hyperparameters 420 may include specified values for the filter size, filter rate, and number of filtering layers. The filter rate represents the number of additional filters added to each successive network layer.

In some embodiments, the MLTM 168 may retrieve the training dataset 430 and the validation dataset 470 from the data store 164 and provide the training dataset 430 to the untrained ML models 410 in a training step. The training dataset 430 propagates forward through the untrained ML models 410, causing the untrained ML models 410 to generate one or more predictions. A prediction may be a time window of the waveform that is labeled as spontaneous systolic, spontaneous diastolic, compression systolic, and/or compression diastolic.

In some embodiments, the MLTM 168 computes a loss and training evaluation metric in block 440 by comparing the predictions generated by the untrained ML models 410 to the ground truth labels in the training dataset 430. For example, the MLTM 168 may apply categorical focal cross-entropy as the loss function and apply intersection over union (IOU) as the training evaluation algorithm. Categorical focal cross-entropy compares the predicted labels (classes) to the ground truth manually applied labels. Categorical focal cross-entropy applies a focal factor to down-weight easier examples and focus more on harder examples. The loss may be backpropagated through the untrained ML model 410 to adjust the weights and biases of the model. The training, which may occur over a plurality, e.g., 25, of epochs, may cause the untrained ML models 410 to adjust model weights so as to minimize the loss function. IOU compares the ground truth labeled time window to the predicted time window for a peak. Specifically, the duration of intersection, e.g., overlap, of the two time windows is divided by the duration of union of the two time windows.

In some embodiments, once training is complete, the best performing, e.g., lowest loss or highest evaluation metric, untrained ML model 410 is selected as the trained ML model.

In some embodiments, the trained ML model 450 may be tuned with a set of tuning hyperparameters 460, such as specified values for the filter size, filter rate, and number of filtering layers.

In some embodiments, the MLTM 168 may calculate a prediction error 480 by comparing the predicted peak time windows to the manually labeled ground truth time windows. The prediction error 480 may include the precision, sensitivity, and/or F1 score at a threshold time interval for the identified systolic peaks and diastolic points. A tolerance of 15 milliseconds may be used to determine correctly located systolic points, and a tolerance of 50 milliseconds may be used to determine correctly located diastolic points, for example. A training administrator or the MLTM 168 may use the prediction error 480 to tune the trained ML model 450 by adjusting one or more tuning hyperparameters 460 to minimize prediction error 480. For example, the optimal tuning hyperparameters 460 may be selected based on the highest F1 score averaged across the four labeling tasks.

In some embodiments, once training and validation are complete, the MLTM 168 may apply model compression 490 to the trained ML model 450 to generate the compressed ML model 132. Model compression 490 may reduce the size of the ML model. Model compression 490 may include quantization, which reduces the precisions of the weights, e.g., from 64-bit floating point to 32-bit floating point, and/or distillation.

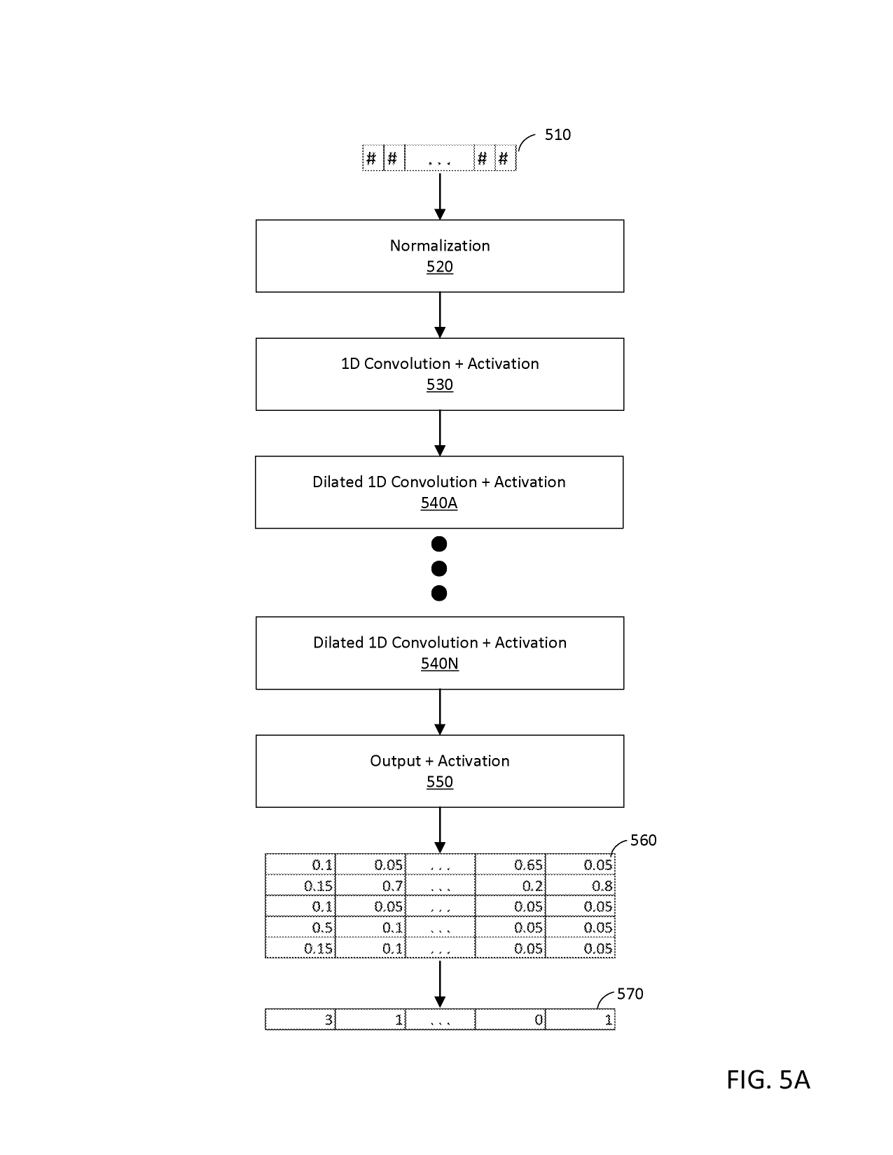

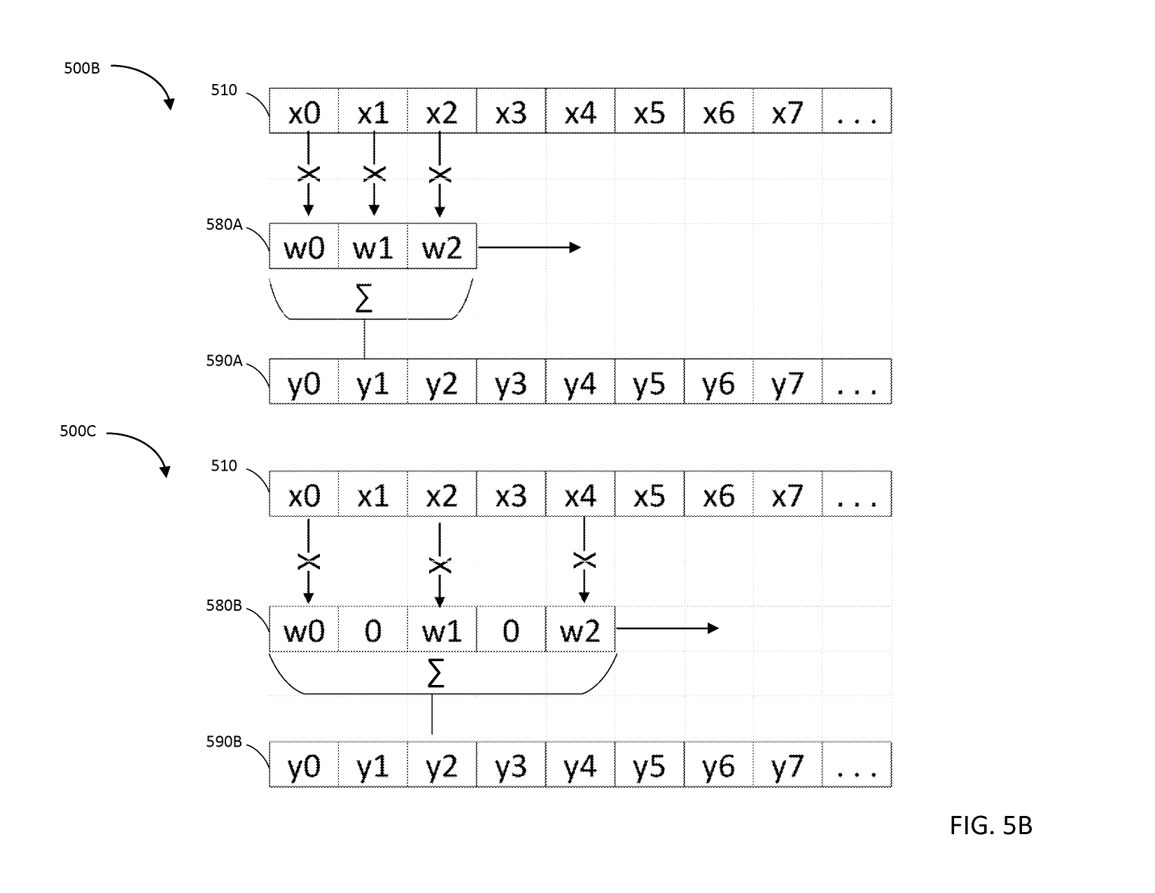

FIG. 5A depicts an exemplary ML model 500A, such as compressed ML model 132, for analyzing arterial pressure waveforms in accordance with various aspects discussed herein. The ML model 500A includes a plurality of layers. The ML model 500A includes an encoder pathway and a decoder pathway. In some embodiments, the encoder pathway includes stacked dilated convolution layers. In some embodiments, the decoder pathway includes a gated recurrent unit (GRU), a dropout layer, and/or a dense output layer.

In some embodiments, input data 510 is provided to the ML model 500A. The input data 510 may comprise a plurality, e.g., 400, of time-ordered data points for a time segment, e.g., two seconds, of arterial pressure data. The data points may represent arterial pressure measurements, such as in millimeters of Hg.

In some embodiments, the ML model 500A includes a normalization layer 520. The normalization layer 520 shifts and scales the data points in the input data 510 into a distribution centered around zero and having a standard deviation of one.

Turning now to FIG. 5B, an exemplary normal one-dimensional (1-D) convolution 500B and an exemplary dilated1-D convolution 500C are illustrated. The input data 510 may be a vector including a plurality of data points x0 – x7.

In the example normal 1-D convolution 500B, the filter 580A includes three weights, w0, w1, and w2. The normal 1-D convolution 500B may perform element-by-element multiplication between the filter 580A and the corresponding input data 510 and sum the products together to generate an element of output data 590A. In the illustrated example, y1 = w0*x0 + w1*x1 + w2*x2. Then the filter 580A may be shifted one or more positions to the right with respect to the input data 510.

In the example dilated 1-D convolution 500C, the filter 580B includes the same three weights, w0, w1, and w2, as the filter 580A. However, the filter 580B has one or more zeros inserted between the weights. In the illustrated example, filter 580B has a dilation rate of two, resulting in one zero between each weight, thus the filter 580B has a receptive window of five. Thus, dilated convolution enables a larger receptive field, so as to “see” more of the pressure waveform data without increasing filter size, which would result in greater computational complexity. The dilated 1-D convolution 500C may perform element-by-element multiplication between the filter 580B and the corresponding input data 510 and sum the products together to generate output data 590B. In the illustrated example, y2 = w0*x0 + w1*x2 + w2*x4. Then the filter 580B may be shifted one or more positions to the right with respect to the input data 510.

Returning to FIG. 5A, in some embodiments, the ML model 500A includes a one-dimensional (1-D) convolution and activation layer 530. The convolution and activation layer 530 may apply a specified number of filters, e.g., 4, 8, 16, 32, etc., of a specified length, e.g., three, to the normalized input data until a feature map is output. The convolution and activation layer 530 may apply a non-linear activation function, e.g., rectified linear unit (ReLU), softmax, sigmoid, hyperbolic tangent, etc., to the feature map.

In some embodiments, the ML model 500A includes a plurality of stacked dilated 1-D convolution and activation layers 540A – 540N. In some embodiments, each successive stacked dilated 1-D convolution and activation layers 540A – 540N applies a larger dilation rate, e.g., 2, 4, 8, 16, 32, and so on, than the prior layer. The plurality of stacked dilated 1-D convolution and activation layers 540A – 540N may apply a non-linear activation function to its feature map.

In some embodiments, the ML model 500A includes an output and activation layer 550. The output and activation layer 550 may perform a final convolution and activation function and generate a probability array 560. The probability array 560 may include a column for each input data point and a row for each blood pressure detection category, e.g. five rows. The probability array 560 includes predicted probabilities of the blood pressure detection categories, with each column summing up to one.

In some embodiments, the ML model 500A converts the probability array 560 into a category vector 570. The category vector 570 may include integers, e.g., 0 – 4, representing the predicted categories for the plurality of data points.

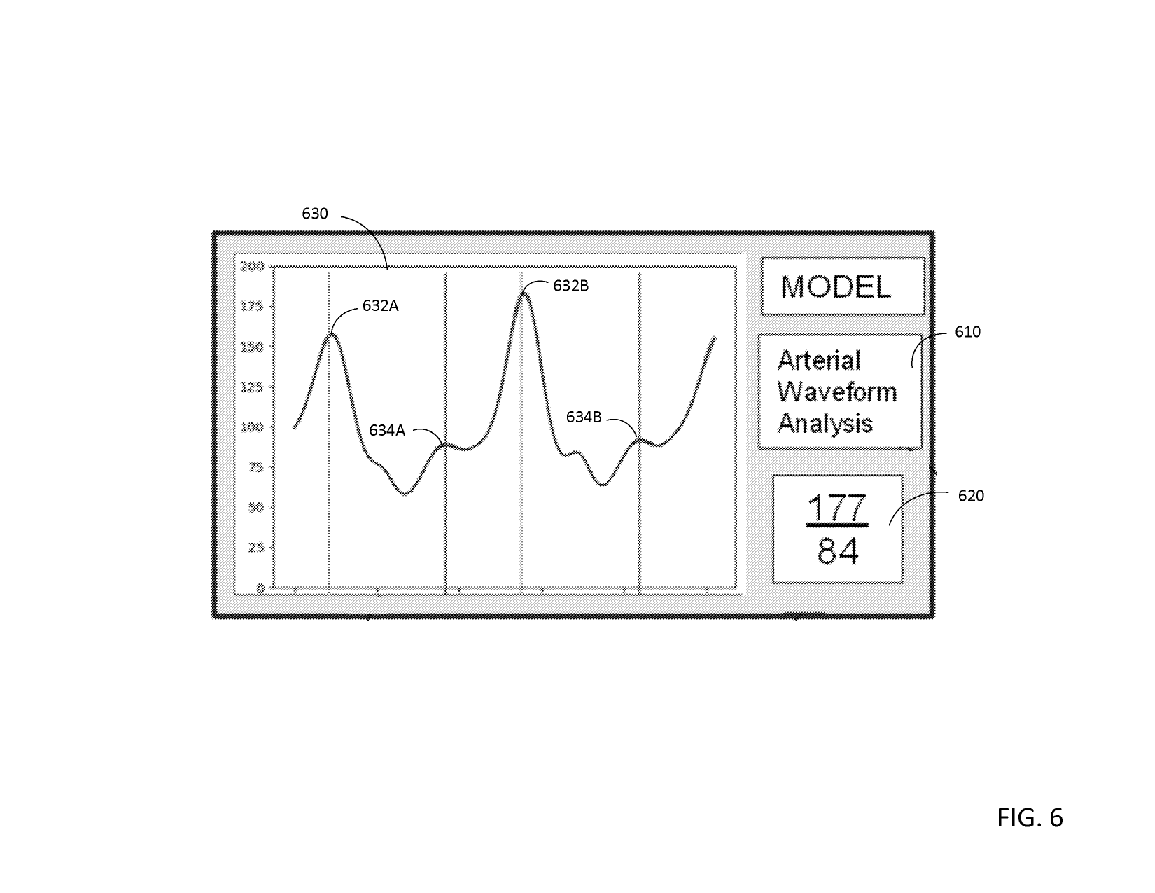

FIG. 6 depicts an example of the display 126 on the blood pressure analyzer 110. In some embodiments, the display 126 includes a touchscreen that enables user input.

In some embodiments, the display 126 includes a mode 610. The mode 610 may indicate what function, e.g., arterial waveform analysis, the blood pressure analyzer 110 is performing. In some embodiments, the display 126 includes a numeric output 620. The numeric output 620 may display values for the systolic and diastolic blood pressures.

In some embodiments, the display 126 includes a waveform output 630. The waveform output may display continuously measured blood pressure versus time. The waveform output may label the predicted systolic points 632A and 632B and predicted diastolic points 634A and 634B for the heartbeat or chest compression cycle.

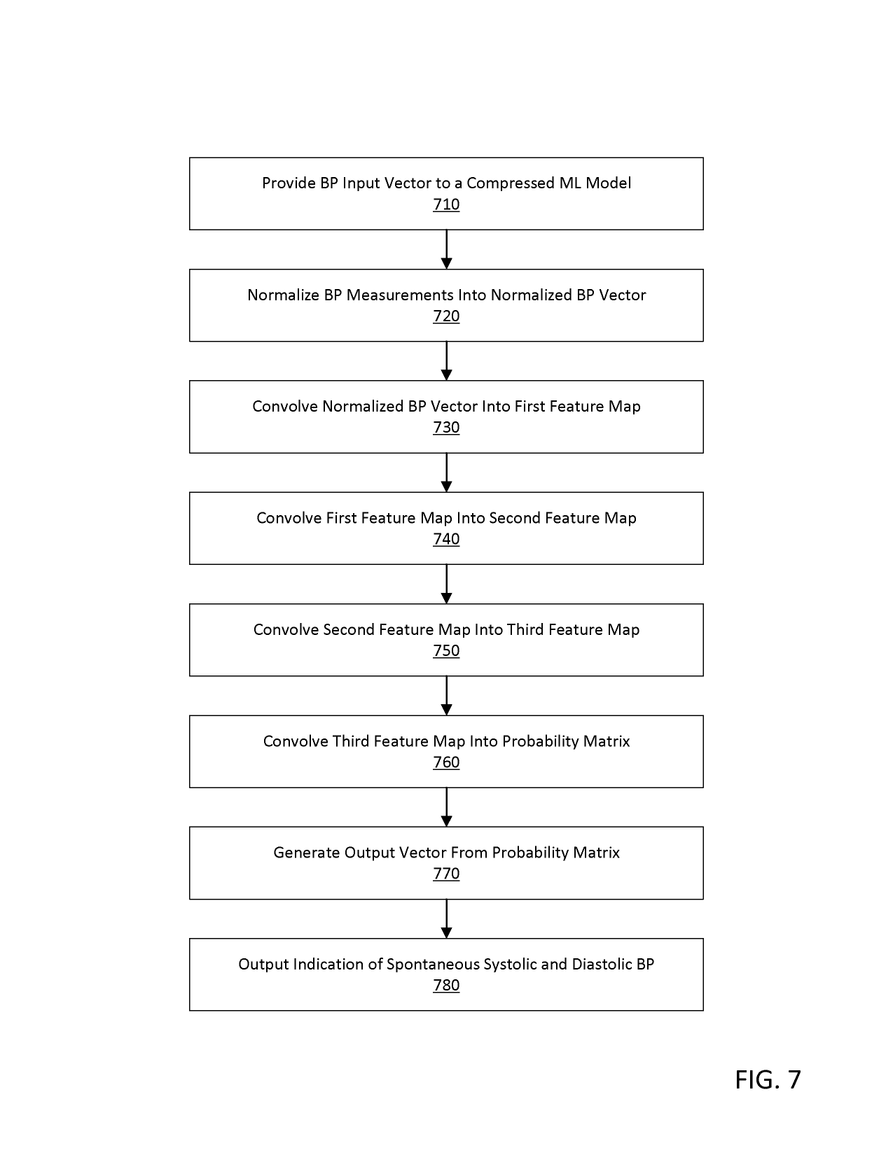

FIG. 7 depicts a flow diagram of an exemplary method 700 for performing arterial pressure waveform analysis, in accordance with various aspects discussed herein. One or more steps of the method 700 may be implemented as a set of instructions stored on a computer-readable memory and executable on one or more processors. The method 700 of FIG. 7 may be implemented via a system, such as the blood pressure analyzer 110, patient monitor 140, and/or chest compression device 142. The method 700 may operate in conjunction with the scenarios and/or environments illustrated in FIGS. 1 – 6 and/or in other environments.

In some aspects, CPR are administered to the patient during the method 700.

In some aspects, the method 700 may include at block 710 providing a blood pressure input vector, such as input data 510, to a compressed ML model, such as compressed ML model 132. The blood pressure input vector may include a plurality of blood pressure measurements, e.g., 400, of a patient during a time period, e.g., two seconds. Providing the blood pressure input vector to the compressed ML model may cause the compressed ML model to perform one or more of blocks 720 – 760. In some embodiments, the compressed ML model comprises a quantized ML model, such as, for example, a quantized FCN model.

In some aspects, the method 700 may include at block 720 normalizing the plurality of blood pressure measurements in the blood pressure input vector into a normalized blood pressure vector.

In some aspects, the method 700 may include at block 730 convolving the normalized blood pressure vector into a first feature map. The compressed ML model may perform the convolution using a first set of one or more filters.

In some aspects, the method 700 may include at block 740 convolving the first feature map into a second feature map. The compressed ML model may perform the convolution using a second set of one or more dilated filters. The second set of one or more dilated filters may be dilated at a first dilation rate.

In some aspects, the method 700 may include at block 750 convolving the second feature map into a third feature map. The compressed ML model may perform the convolution using a third set of one or more dilated filters. The third set of one or more dilated filters may be dilated at a second dilation rate that is greater than the first dilation rate. In some embodiments, the second dilation rate is double the first dilation rate. In some embodiments,

In some aspects, the method 700 may include at block 760 convolving the third feature map into a probability matrix. The compressed ML model may perform the convolution using a fourth set of one or more dilated filters. In some embodiments, the first set of one or more filters, the second set of one or more dilated filters, the third set of one or more dilated filters, and the fourth set of one or more dilated filters comprise filters comprising an equal filter size. In some embodiments, the equal filter size is three. The probability matrix may comprise probabilities for a plurality of classifications for one or more of the plurality of blood pressure measurements. In some embodiments, the plurality of classifications comprise spontaneous systolic pressure and spontaneous diastolic pressure. In some embodiments, the plurality of classifications further comprise compression systolic pressure, compression diastolic pressure, and nothing.

In some aspects, the method 700 may include at block 770 generating an output vector from the probability matrix. The output vector may comprise indications, e.g., integers such as 0 - 4, of predicted classifications for the one or more of the plurality of blood pressure measurements.

In some aspects, the method 700 may include at block 780 outputting an indication of the spontaneous systolic pressure and the spontaneous diastolic pressure to a user. In some embodiments, the compression systolic pressure and the compression diastolic pressures are also output.

In some aspects, responsive to determining that the compression systolic pressure is less than a specified threshold value, the method 700 may include administering a vasopressor, such as epinephrine or norepinephrine. In some aspects, responsive to determining that the compression systolic pressure is less than a specified threshold value, the method 700 may include moving the administration of chest compressions from a first location to a second location.

It should be understood that not all blocks of the exemplary method 700 are required to be performed. Moreover, the method 700 is not mutually exclusive (i.e., block(s) from exemplary flow diagram 700 may be performed in any particular implementation).

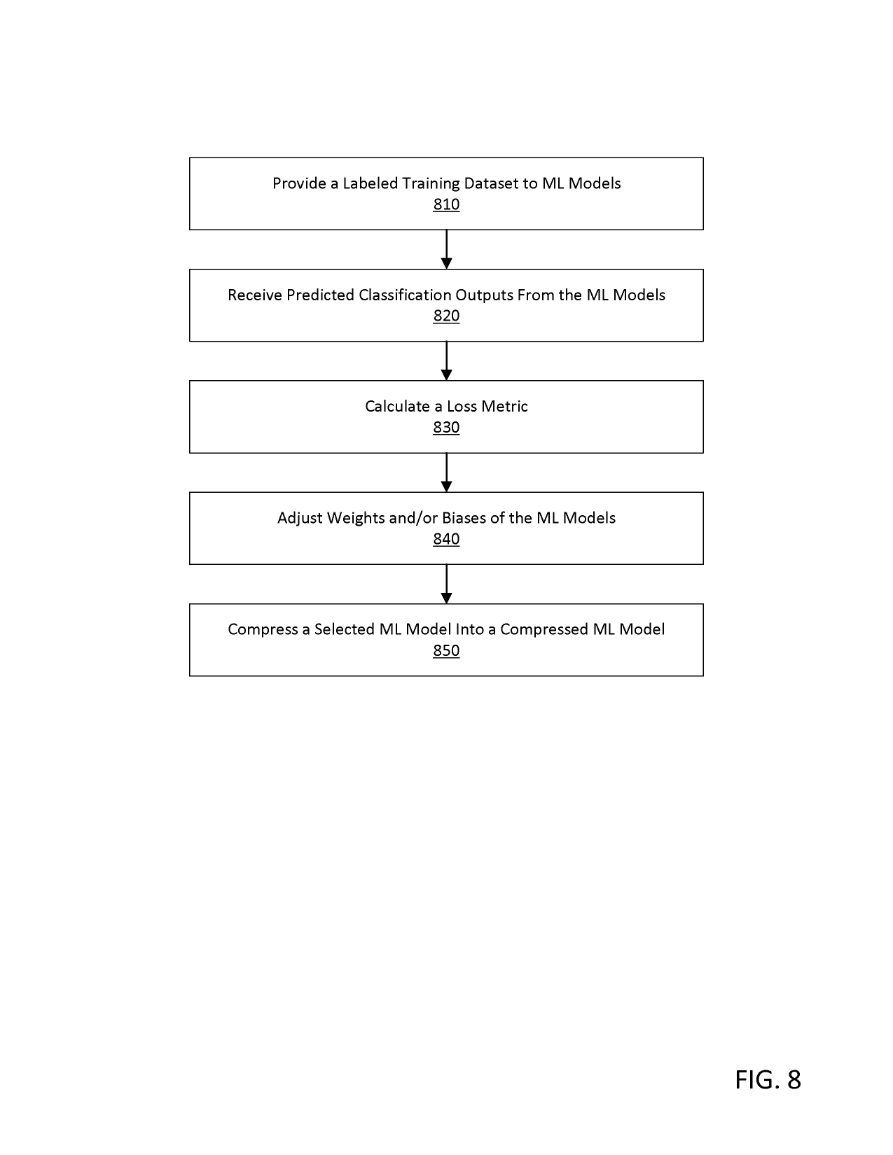

FIG. 8 depicts a flow diagram of an exemplary method 800 for training a ML model to generate blood pressure predictions, in accordance with various aspects discussed herein. One or more steps of the method 800 may be implemented as a set of instructions stored on a computer-readable memory and executable on one or more processors. The method 800 of FIG. 8 may be implemented via a system, such as the training server 150. The method 800 may operate in conjunction with the scenarios and/or environments illustrated in FIGS. 1-7 and/or in other environments.

In some aspects, the method 800 may include splitting a blood pressure dataset into a training dataset and a validation dataset. Splitting the blood pressure dataset may comprise applying k-fold cross validation.

In some aspects, the method 800 may include at block 810 providing a labeled training dataset to one or more ML models. The labeled training dataset may comprise a plurality of blood pressure data points and a plurality of labeled classifications. For example, the plurality of labeled classifications may include spontaneous systolic pressure, spontaneous diastolic pressure, compression systolic pressure, compression diastolic pressure, and/or nothing. As another example, the plurality of labeled classifications may include systolic pressure and diastolic pressure.

In some aspects, the method 800 may include at block 820 receiving predicted classification outputs from the one or more ML models.

In some aspects, the method 800 may include at block 830 calculating a loss metric by comparing the predicted classification outputs to the plurality of labeled classifications. For example, calculating the loss metric may comprise applying categorical focal cross-entropy.

In some aspects, the method 800 may include at block 840 adjusting, based on the loss metric, one or more weights and/or biases of the one or more ML models. Adjusting the weights and/or biases may comprise applying backpropagation to reduce the loss metric.

In some aspects, the method 800 may include calculating a training evaluation metric by comparing the predicted classification outputs to the plurality of labeled classifications. The method 800 may further include selecting the selected one of the one or more ML models based on the training evaluation metric. The training evaluation metric may comprise intersection over union, for example.

In some aspects, the method 800 may include evaluating, using the labeled validation dataset, a performance of the selected ML model. Evaluating the performance may include determining a precision metric, an accuracy metric, or an F1 score

In some aspects, the method 800 may include at block 850 compressing a selected one or the one or more ML models into a compressed ML model.

It should be understood that not all blocks of the exemplary method 800 are required to be performed. Moreover, the method 800 is not mutually exclusive (i.e., block(s) from exemplary flow diagram 800 may be performed in any particular implementation).

### ADDITIONAL CONSIDERATIONS

Although the preceding text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical.

1. A computer-implemented method of determining arterial blood pressure and related properties, comprising:
providing, by one or more processors, a blood pressure input vector comprising a plurality of arterial blood pressure measurements of a patient during a time period to a machine learning model, causing the machine learning model to:
normalize the plurality of arterial blood pressure measurements into a normalized blood pressure vector,
convolve, using a first set of one or more filters, the normalized blood pressure vector into a first feature map,
convolve, using a second set of one or more dilated filters, the first feature map into a second feature map, wherein the second set of one or more dilated filters are dilated at a first dilation rate,
convolve, using a third set of one or more dilated filters, the second feature map into a third feature map, wherein the third set of one or more dilated filters are dilated at a second dilation rate that is greater than the first dilation rate,
convolve, using a fourth set of one or more dilated filters, the third feature map into a probability matrix, wherein the probability matrix comprises probabilities for a plurality of classifications for one or more of the plurality of arterial blood pressure measurements, and
generate, from the probability matrix, an output vector comprising indications of predicted classifications for the one or more of the plurality of arterial blood pressure measurements; and
outputting, by the one or more processors based on the output vector, an indication of thearterial blood pressure to a user.

2. The computer-implemented method of claim 1, wherein cardiopulmonary resuscitation (CPR) are administered to the patient during the time period.

3. The computer-implemented method of claim 1, wherein the machine learning model is a compressed fully convolutional network (FCN) model.

4. The computer-implemented method of claim 1, wherein the second dilation rate is double the first dilation rate.

5. The computer-implemented method of claim 3, wherein the compressed FCN model comprises a quantized FCN model.

6. The computer-implemented method of claim 1, wherein the first set of one or more filters, the second set of one or more dilated filters, the third set of one or more dilated filters, and the fourth set of one or more dilated filters comprise filters comprising an equal filter size.

7. The computer-implemented method of claim 6, wherein the equal filter size is three.

8. The computer-implemented method of claim 1, wherein a duration of the time period is two seconds.

9. The computer-implemented method of claim 1, further comprising:
responsive to determining that the arterial blood pressure is less than a specified value, administer epinephrine, norepinephrine, vasopressin, or other vasoactive medications to the patient.

10. The computer-implemented method of claim 1, further comprising:
responsive to determining that the arterial blood pressure is less than a specified value, move an administration of chest compressions from a first location to a second location.

11. A portable computing device for determining arterial blood pressure and related properties, comprising:
one or more processors; and
one or more non-transitory memories coupled to the one or more processors and storing instructions that when executed by the one or more processors, cause the one or more processors to:
provide a blood pressure input vector comprising a plurality of arterial blood pressure measurements of a patient during a time period to a machine learning model, causing the machine learning model to:
normalize the plurality of blood pressure measurements into a normalized blood pressure vector,
convolve, using a first set of one or more filters, the normalized blood pressure vector into a first feature map,
convolve, using a second set of one or more dilated filters, the first feature map into a second feature map, wherein the second set of one or more dilated filters are dilated at a first dilation rate,
convolve, using a third set of one or more dilated filters, the second feature map into a third feature map, wherein the third set of one or more dilated filters are dilated at a second dilation rate that is greater than the first dilation rate,
convolve, using a fourth set of one or more dilated filters, the third feature map into a probability matrix, wherein the probability matrix comprises probabilities for a plurality of classifications for arterial blood pressure measurements, and
generate, from the probability matrix, an output vector comprising indications of predicted classifications for the arterial blood pressure measurements, and
output, based on the output vector, an indication of the arterial blood pressure to a user.

12. The portable computing device of claim 11, wherein cardiopulmonary resuscitation (CPR) is administered to the patient during the time period.

13. The portable computing device of claim 11, wherein the machine learning model is a compressed fully convolutional network (FCN) model.

14. The portable computing device of any one of claim 11, wherein the second dilation rate is double the first dilation rate.

15. The portable computing device of any one of claim 13, wherein the compressed FCN model comprises a quantized FCN model.

16. The portable computing device of any one of claim 11, wherein the first set of one or more filters, the second set of one or more dilated filters, the third set of one or more dilated filters, and the fourth set of one or more dilated filters comprise filters comprising an equal filter size.

17. The portable computing device of claim 16, wherein the equal filter size is three.

18. The portable computing device of any one of claim 11, wherein a duration of the time period is two seconds.

19. The portable computing device of claim 11, further comprising:
a display,
wherein outputting the indication of the arterial blood pressure comprises outputting on the display.

20. A computer-implemented method of training a machine learning model to predict arterial blood pressure, comprising:
providing, by one or more processors, a labeled training dataset to one or more machine learing models or other processing methods, wherein the labeled training dataset comprises a plurality of blood pressure data points and a plurality of labeled classifications;
receiving, by the one or more processors, predicted classification outputs from the one or more machine learning models;
calculating, by the one or more processors, a loss metric by comparing the predicted classification outputs to the plurality of labeled classifications;
adjusting, by the one or more processors using the loss metric, one or more weights and/or biases of the one or more machine learning models; and
compressing, by the one or more processors, a selected one of the one or more machine learning models into a compressed machine learning model.
